(19) United States
(12) Patent Application Publication
(10) Pub. No.: US 2018/0147082 A1
(43) Pub. Date: May 31, 2018

(54) Flexible nonviolent intervention shield

(71) Applicant: Harold Maltais, McLeods (CA)

(72) Inventor: Harold Maltais, McLeods (CA)

(21) Appl. No.: 15/530,051

(22) Filed: Nov. 28, 2016

(51) Int. Cl. A61F 5/37

(52) U.S. Cl. CPC A61F 5/37

(57) ABSTRACT

The nonviolent shield is used for restraining an agitated standing individual, while protecting that individual from a violent fall or similar jerking associated with a restraining intervention. The nonviolent shield comprises a rectangular flexible cargo net; an elongated body-height central cushion attached to the cargo net along a median of the cargo net, and two elongated body-height side cushions attached to the cargo net in a spaced-apart parallel relationship with the central cushion. The present nonviolent shield also has handhold areas of the cargo net bordering the central cushion. In another aspect, the central cushion has a movable portion on an upper segment thereof so that the central cushion is brought with firmness against the torso of the individual to be restrained, and with lightness against the face and head of that individual.

### FIELD OF THE INVENTION

This invention pertains to restraining shields and more particularly it pertains to restraining shields offering protection to both the interveners and the person being confronted.

### BACKGROUND OF THE INVENTION

Intervention shields are used in mental institutions, in prisons, or by riot police officers to bring an aggressive person under submission. These shields are often used with force. While the shields of the prior art have handles on one side thereof for use by the interveners, the other surface does not offer any handhold feature for use by the person being subdue, to limit the extent of injuries to that person during a fall for example.

Examples of shields found in the prior art include the devices described in the following publications:

U.S. Pat. No. 2,703,412, issued to D. J. Stone on Mar. 8, 1955;

U.S. Pat. No. 2,766,751, issued to Peter Topa on Oct. 16, 1956;

U.S. Pat. No. 3,399,670, issued to J. K. Veasey on Sep. 3, 1968;

U.S. Pat. No. 3,476,107, issued to A. R. Matt Jr. Et al., on Nov. 4, 1969;

U.S. Pat. No. 4,186,453, issued to J. E. Burns on Feb. 5, 1980;

U.S. Pat. No. 4,852,587, issued to Mark Share on Aug. 1, 1989;

U.S. Pat. No. 5,111,850, issued to M. I. Kunofsky on May 12, 1992;

U.S. Pat. No. 5,329,636, issued to Bruce K. Siddle on Jul. 19, 1994;

U.S. Pat. No. 6,367,427, issued to Duane C. Canady on Apr. 9, 2002;

U.S. Pat. No. 6,491,038, issued to A. A. McQueen on Dec. 10, 2002;

CA Patent 2,275,452, issued to R. Widmont on May 9, 2000;

EP Patent Appl. 0 255 761 filed by E. N. Bauer on Aug. 7, 1987;

EP Patent Appl. 2 037 207 filed on Oct. 9, 2008 by E. B. Christensen.

In many cases of violent behaviour, the person being considered violent is often a person depressed experiencing an episode of anxiety. Such episodes are often caused by a recent crisis in his/her life. An abnormal mental behaviour can also be caused by a vitamin deficiency, long-enduring pain or other medical issue beyond the control of the person being considered abnormal. Therefore, it is believed that an abnormal behaviour is not a sufficient cause to treat a person with brutality.

It is also believed that there is a need in mental institutions and policing tools for an intervention shield that can be used in a nonviolent manner to subdue a person while protecting that person from injuries associated with such interventions.

### SUMMARY OF THE INVENTION

In the present invention, there is provided a nonviolent intervention shield for restraining an agitated standing individual, while protecting that individual from a fiery fall or similar shock associated with a restraining intervention.

In a first aspect of the present invention, there is provided a nonviolent intervention shield comprising: a rectangular flexible cargo net; a central body-height elongated cushion attached to the cargo net along a median of the cargo net, and two body-height elongated side cushions attached to the cargo net in a spaced-apart, parallel relationship with the central cushion, one along each side of the central cushion. The present nonviolent intervention shield also has meshes and strands offering handhold areas on the cargo net bordering the central cushion.

During an intervention, the central cushion and the handhold areas are brought first in contact with the individual to be restrained, so that the individual can readily grab the cargo net and restrain himself/herself from a fall.

In another aspect of the present invention, the central cushion has a movable portion on an upper end thereof so that the central cushion is brought with firmness against the torso of the individual to be restrained, and with lightness against the face and head of that individual.

In yet another aspect of the present invention, there is provided a method for restraining an agitated standing individual. This method comprises the steps of:
providing a flexible shield comprising a body-height central cushion and two body-height side cushions attached to a cargo net in a spaced-apart, parallel relationship from each other;
mounting the central cushion along a median of the cargo net;
mounting the side cushions along the cargo net in such a way as to provide handhold areas on the cargo net on each side of the central cushion;
confronting the individual with all three cushions positioned side-by-side close to each other and;
moving the flexible shield toward the individual while deploying the cargo net sideways for separating the cushions from each other and exposing the handhold areas;
moving the handhold areas against the hands of the individual while moving the central cushion against the torso of the individual;
wrapping the cargo net around the individual;
moving the side cushions against the shoulders and back of the individual, and
pressing all three cushions against the torso, shoulders and back of the individual, respectively.

This method offers handhold support to the individual to be restrained, while protecting his/her face, head, shoulder and back from the jerks of a fall or any other abrupt manipulation of the shield.

This brief summary has been provided so that the nature of the invention may be understood quickly. A more complete understanding of the invention can be obtained by reference to the following detailed description of the preferred embodiment thereof in connection with the attached drawings.

### BRIEF DESCRIPTION OF THE DRAWINGS

A preferred embodiment of the nonviolent intervention shield according to the present invention and method of use are described with the aid of the accompanying drawings, in which like numerals denote like parts throughout the several views, and in which:

FIG. 1 is a perspective view of a nonviolent shield according to the preferred embodiment of the present invention, being used by two attendants to subdue a distressed patient;

FIG. 2 is a second perspective view of the preferred nonviolent shield with the elongated cushions being used in an alternate configuration;

FIG. 3 illustrates a preferred positioning of the hands of an attendant for manipulating the preferred nonviolent shield during an intervention;

FIG. 4 is a plan view of a preferred configuration of the preferred nonviolent shield during a first step in an intervention;

FIG. 5 is the plan view of a preferred configuration of the preferred nonviolent shield during a second step in an intervention;

FIG. 6 is a plan view of a preferred configuration of the preferred nonviolent shield during a third step in an intervention;

FIG. 7 is an enlarged partial plan view of a preferred configuration of the preferred nonviolent shield during a fourth step in an intervention;

FIG. 8 illustrates a front elevation view of the preferred nonviolent shield;

FIG. 9 illustrates a rear elevation view of the preferred nonviolent shield;

FIG. 10 shows an aggregate view of the elements of the nonviolent shield according to the preferred embodiment of the present invention;

FIG. 11 illustrates the preferred nonviolent shield in a stowed mode.

### DESCRIPTION OF THE PREFERRED EMBODIMENT

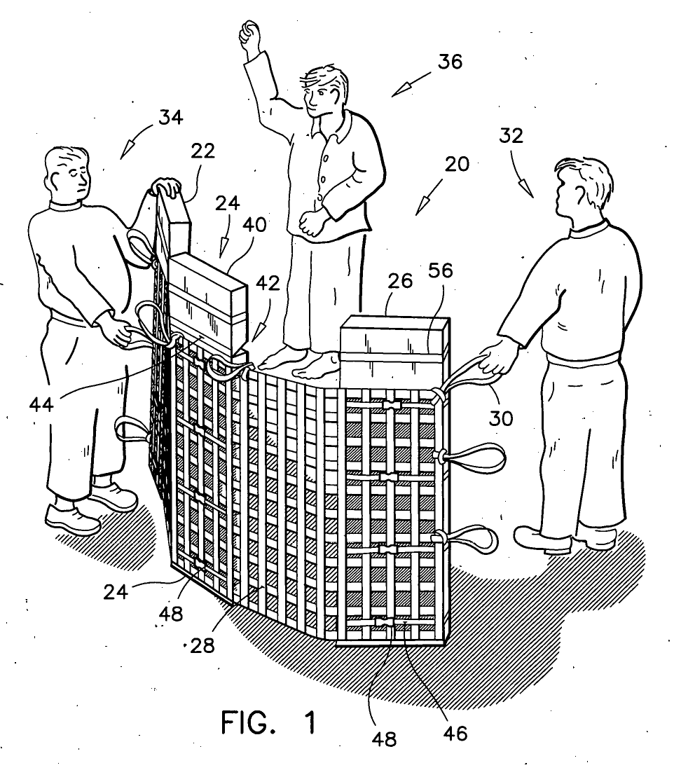

Referring to FIG. 1, the nonviolent intervention shield 20, is made of three vertically elongated, spaced-apart, body-height cushions 22, 24 and 26, attached to a flexible cargo net 28. The expression “body-height” is used herein to designate a sufficient height to cover an average-height individual from head to feet. In a preferred embodiment the expression “body-height” represents a height of about between 50 and 70 inches.

The central cushion 24 is attached to the cargo net 28 along a median of the cargo net 28. Handle-like straps 30 are preferably attached to the cargo net 28 at convenient locations around the cargo net 28. These handle straps 30 are referred to herein as handles of a first type 30, or first-type handles 30. The preferred nonviolent shield 20 is preferably used by two attendants 32, 34 to encircled a person 36 in distress for example, and to bring this person under control.

The preferred cargo net 28 is made of straps, but round ropes can also work. The preferred cargo net 28 has a mesh size that allows for a person to easily grab into the meshes of the cargo net and hold on to one or more strands of the cargo net 28. For reference purposes, the cargo net 28 has a preferred mesh size of about one inch openings. As can be appreciated, some of the drawings are not to scale and were done this way for convenience. The cargo net 28 has a preferred height of 46 inches, a preferred width of about 69 inches, and a preferred spacing between the cushions 22, 24, 26 is about 16 inches.

Although first-type handles 30 are illustrated and described, the nonviolent shield can be used without such handles, by manipulating the nonviolent shield by the meshes and strands of the cargo net.

The expression “cargo net” is used herein to designate the main element in the preferred nonviolent shield 20. This cargo net is made of flat nylon or fabric strands sewn together and forming square meshes. The cargo strap can also be made of ropes tied to form square meshes, or diamond-shaped meshes. These cargo net are used as crane-lifting bags for handling wood crates, merchandise on pallets, grain bags, etc., when loading or unloading a ship for example. These cargo nets are also used to transport camp supplies by helicopter, or to cover a load of firewood in a truck box.

The body-height cushions 22, 24, 26 have durable leather-like casings made of plastic or fabric. The casings are filled with foam of the type used in upholstery or in mattresses. The preferred dimensions for the cushions are a width of about 12 inches, a height of about 60 inches, as mentioned before, and a thickness of about 2 inches.

The central cushion 24 has a removable upper segment 40. This removable segment 40 is movable about a recess 42 in the cushion 24. This movable segment 40 can be moved forward and back from the plane of the cushion 24, about the recess 42. The removable upper segment 40 is held to the lower segment of the central cushion 24 by a strip of hook-and-loop fasteners 44. The strip of hook-and-loop fasteners 44 constitutes a hinge. The preferred dimensions for the movable segment 40 is 12 inch wide by 14 inch high.

Each body-height cushion 22, 24, or 26 is attached to the cargo net 28 by means of a series of cushion straps 46 extending through the meshes of the cargo net 28, and cushion buckles 48 on these cushion straps 46. Preferably, each cushion 22, 24 or 26 is attached to the cargo net 28 by means of four cushion straps 46 and cushion buckles 48. The cushion straps are made of nylon or fabric.

It will be appreciated from the illustration of FIG. 1 that when a person is being encircled by the preferred nonviolent shield 20, the central cushion 24 is brought directly in front of that person, while the side cushions 22, 26 are brought against the shoulders of that person. This method, in combination with the configuration of the preferred shield 20 allow the person being subdue to grab the cargo net 28 on each sides of the central cushion 24 and to hold him/herself to the cargo net 28 while being immobilized for example. The ability to hold oneself to the cargo net 28 offers a certain degree of security wherein that person being confined inside the shield 20 is not completely helpless in preventing an injury from a fall to the ground for example.

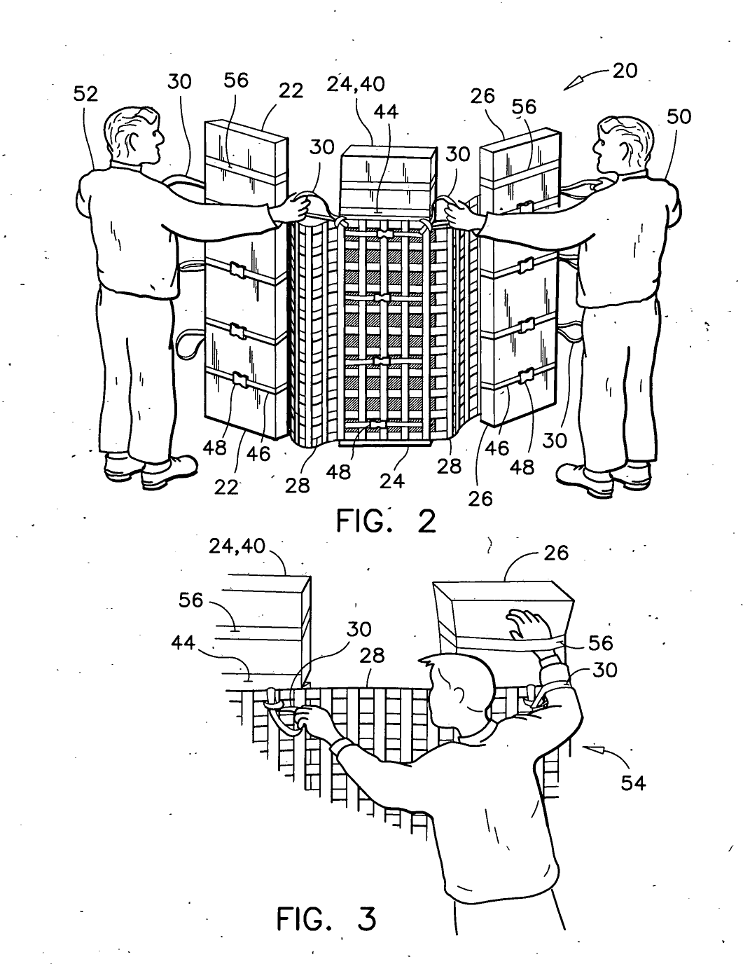

Referring now to FIG. 2 the preferred nonviolent shield 20 is shown with the central cushion 24 mounted to the cargo net 28 on the inside side of the cargo net 28, and the side cushions 22, 26 being mounted to the cargo net 28 on the outside side of the cargo net 28, facing the attendants 50, 52 handling the preferred nonviolent shield 20. Because the cushions 22, 24, and 26 are mounted to the cargo net 28 with cushion straps 46 and cushion buckles 48, and the straps 46 are relatively long, these cushions can be mounted on either side of the cargo net 28, to offer protection to either the attendants 50, 52 or to the person being subdue.

A preferred hand position by an attendant 54 holding the preferred nonviolent shield is suggested in FIG. 3. As can be seen in most of the accompanying drawings, the upper portion of each cushion has a handle strap 56 there-across. These straps 56 are referred to herein as handles of a second type 56, or second-type handles 56. In use, an attendant inserts one arm into a first-type handle 30 closest to the upper portion of one of the outside cushions, 26 for example as illustrated in FIG. 3 and grabs or inserts his/her hand under one of the second-type handle 56 on that cushion to acquire a good control over the movement of that cushion 26 during an intervention. The other hand can be used in a same way to control the movement of the central cushion, or to hold on to a first-type handle 30 near the center of the preferred nonviolent shield 20. It will be appreciated that the preferred hand placement by the other attendant is a mirror image of the illustration in FIG. 3.

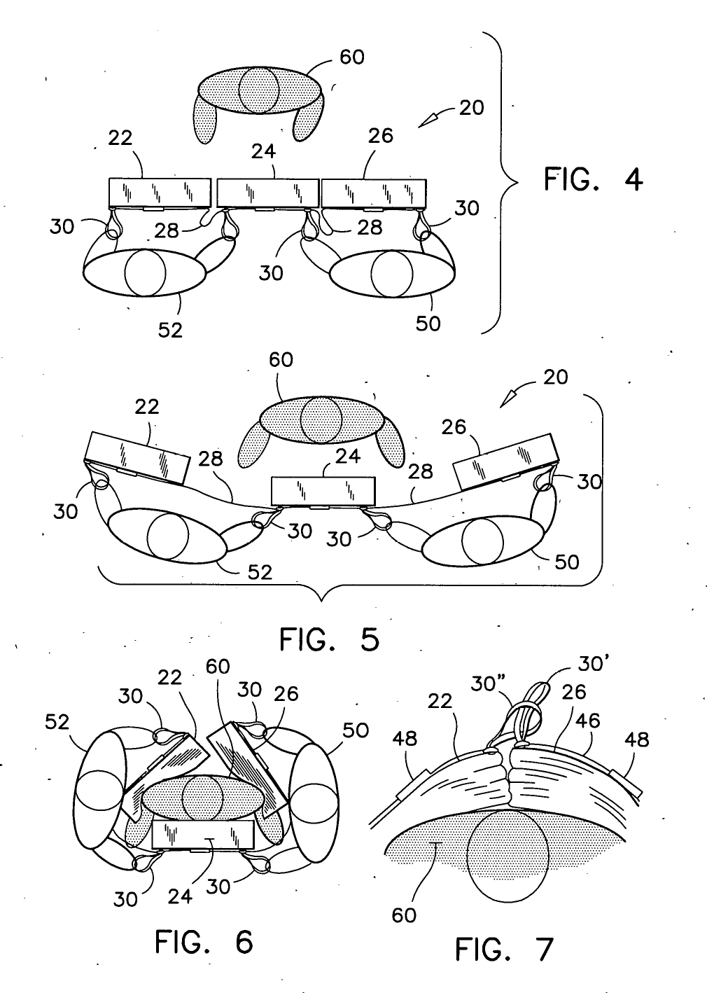

FIGS. 4 to 7 illustrate a preferred method for carrying an intervention on a patient 60, using the preferred nonviolent shield 20 according to the preferred embodiment of the present invention. An intervention is initiated by confronting a patient 60 with the three cushions 22, 24 and 26 of the preferred nonviolent shield 20 being held closed to each other, forming a straight wall, as illustrated in FIG. 4.

As the patient 60 is approached, the two attendants 50, 52 quickly deploy the preferred nonviolent shield 20 by expanding the cargo net 28, placing the central cushion 24 against the torso of the patient 60, and positioning the side cushions 22 and 26, as far apart from each other as possible, while encircling the patient 60, as illustrated in FIGS. 5 and 6.

When referring to FIG. 6, an important feature of the preferred nonviolent shield 20 can be seen. In this illustration, the person being restrained 60 has a first cushion 24 pressed against his/her torso, and two side cushions 22, 26 pressed against his/her shoulders and back. His/her hands, however are held by the portions of cargo net 28 between the cushions. The person being restrained 60 can hold himself/herself to the cargo net 28 during the entire intervention. The width of cargo net 28 between the central cushion 24 and one of the side cushions 22, 26 is about 16 inches, providing unobstructed handhold areas between the cushions, and sufficient distance for encircling a person 60; as illustrated.

If ever the person to be restrained 60 falls during an intervention, the first cushion 24 protects his/her face and torso; the second and third cushions 22, 26 protect his/her shoulders and back. This triangular configuration of the preferred nonviolent shield 20 in use prevents, to a great extent, injuries to the person 60 to be restrained.

When the person 60 to be restrained is encircled by the preferred nonviolent shield 20, as illustrated in FIG. 7, two or more first-type handles 30′ on one side of the nonviolent shield 20 can be inserted into corresponding first-type handles 30″ on the other side of the nonviolent shield 20 to lock the nonviolent shield 20 around the person 60 to be restrained. That individual can then be lowered down to a stretcher and sedated for example.

When the upper portion 40 of the central cushion 24 makes the person 60 being restrained uncomfortable, that upper portion 40 can be folded about the hinge 44 away from the individual's face or removed altogether by undoing the strip of hook and loop fastener 44. The mounting of the upper portion 40 on hinge 44 provides flexibility in the central cushion 24.

Referring back to FIG. 5, it will be appreciated that when a person 60 to be restrained is confronted as described earlier with the preferred nonviolent shield 20, the natural instinct of that person causes him/her to grab the cargo net 28 on each side of the central cushion 24. This natural movement keeps the nonviolent shield 20 centered on that individual 60, facilitating the wrapping movement of the side cushions 22, 26 by the attendants 50, 52. The symmetrical structure of the nonviolent shield 20 does not offer any incentive to the person 60 to pull the shield sideway to escape the intervention. The symmetrical structure of the preferred nonviolent shield 20, encourages the person 60 to be restrained to remain centered in the nonviolent shield 20, facilitating the work of the attendants 50, 52.

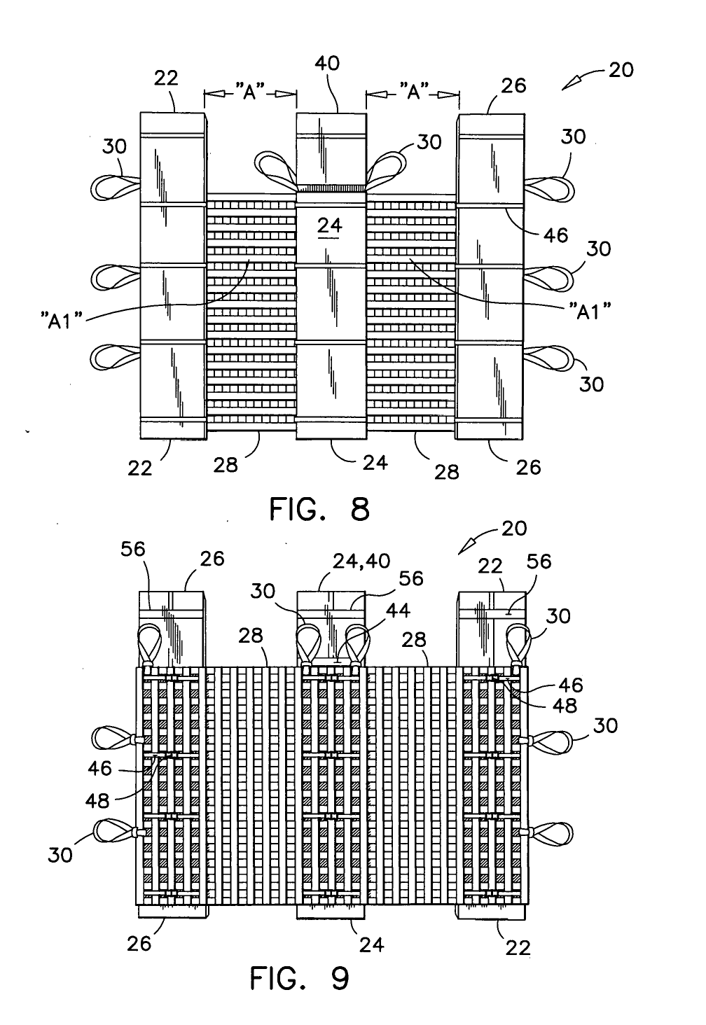

FIG. 8 of the drawings illustrates a front view of the preferred nonviolent shield as seen by the individual 60 to be restrained. As can be imagined, the open-mesh portions “A” of the cargo net 28 represent, to that individual, available handhold area “A1”.

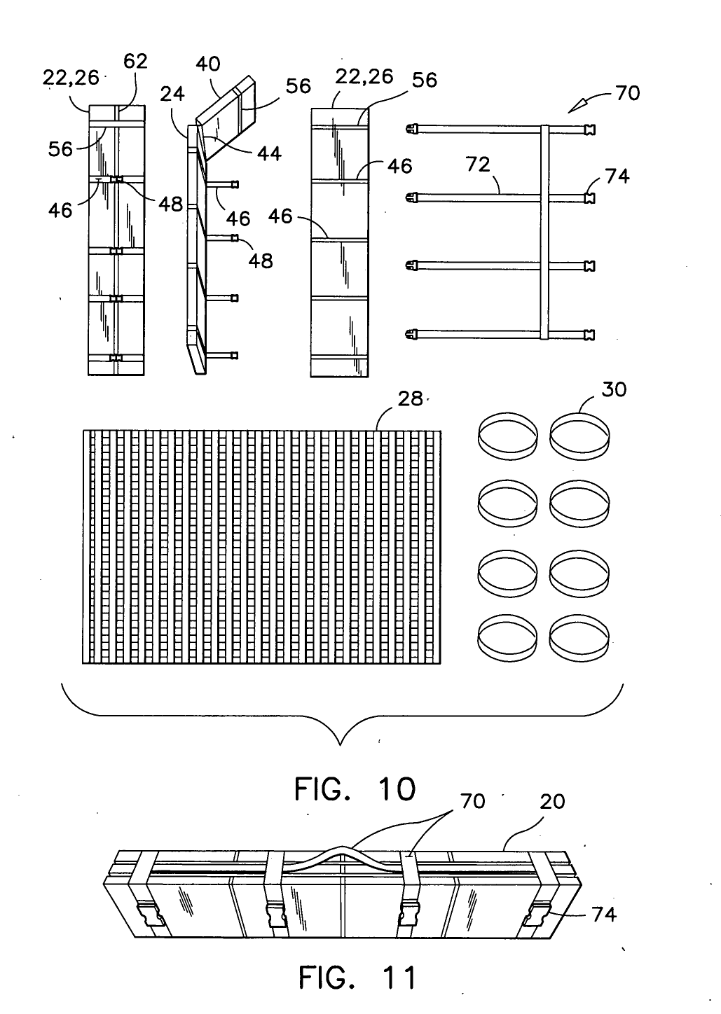

Referring now to FIGS. 9 and 10, the cushions 22, 24 and 26 have cushion straps 46 and cushion buckles 48 permanently attached to them. These cushion straps 46 are used to attach the cushions to the cargo net 28, on one side of the cargo net 28 or the other. The cushions 22, 24, and 26 are attached to the cargo net 28 so that the bottom ends of the cushions rest below the lower edge of the cargo net 28, as illustrated in FIG. 9.

As it was previously explained, the central cushion 24 is attached to the median of the cargo net 28, and the outside cushions 22, 26 are attached to the vertical outside edges of the cargo net 28, in such a way as to leave an open-mesh portion “A” of about 16 inches wide on each side of the central cushion 24.

For convenience, each of the cushions 22, 24 and 26 has a zippered opening 62 along the full height thereof to facilitate their manufacture.

The first-type handles 30 are preferably made of flexible flat nylon or fabric straps of about 1 inch wide. Other strong material can also be used, according to the preference of the manufacturer. These handles 30 are formed in closed loops as illustrated in FIG. 10, and can be attached to the cargo net 28 by a Cow Hitch™ knots, for example, anywhere to the cargo net 28. Preferably, three first-type handles 30 are tied to each of the vertical outside edges of the cargo net 28 and two first-type handles 30 are attached to the upper edge of the cargo net 28, one on each side of the central cushion 24.

FIG. 10 also illustrates a harness strap assembly 70, which is made of harness straps 72 and harness buckles 74. This harness strap assembly 70 is used to tie the preferred nonviolent shield 20 in a compact form for storage. The compact package as illustrated in FIG. 11, comprises all three juxtaposed cushions 22, 24, and 26, with the cargo net 28 and first-type handles 30 folded between the cushions.

The preferred nonviolent shield 20 is easily deployed for use by undoing the harness buckles 74 and opening the nonviolent shield 20 as one would open a book.

As to other details and manner of operation of the preferred nonviolent shield 20, the same should be apparent from the above description and drawings, and accordingly further discussion related to these aspects is deemed unnecessary.

What is claimed is:

1. A nonviolent shield for restraining an individual, comprising:
a rectangular flexible cargo net;
an elongated central cushion attached to said cargo net along a median of said cargo net;
two elongated side cushions attached to said cargo net in a spaced-apart parallel relationship with said central cushion, one along each side of said central cushion; and
handhold areas of said cargo net bordering said central cushion.

2. The nonviolent shield as claimed in claim 1, further comprising a plurality of first-type handles attached to said cargo net.

3. The nonviolent shield as claimed in claim 1, wherein said central cushion has a movable portion on an upper end thereof;

4. The nonviolent shield as claimed in claim 2, wherein each of said cushions has a second-type handle on an upper portion thereof.

5. The nonviolent shield as claimed in claim 2, wherein each of said first-type handles are attached to said cargo net by a Cow Hitch™ knot.

6. The nonviolent shield as claimed in claim 2, further comprising a harness for optionally retaining said cushions, said cargo net and said first-type handles in a compact form.

7. The nonviolent shield as claimed in claim 1, wherein said handhold areas comprises open meshes and strands for grab by said individual.

8. The nonviolent shield as claimed in claim 1, wherein said cushions are removably attached to said cargo net by straps and buckles.

9. The nonviolent shield as claimed in claim 8, wherein said straps and buckles are permanently fastened to said cushions.

10. The nonviolent shield as claimed in claim 8, wherein said straps are configured to attach said cushions to either sides of said cargo net.

11. The nonviolent shield as claimed in claim 3, wherein said central cushion further comprises a hinge and said movable portion is removably attached to said central cushion along said hinge.

12. A nonviolent shield restraining hands, torso, shoulders and back of a standing individual, comprising:
a rectangular flexible cargo net;
an elongated body-height central cushion attached to said cargo net along a median of said cargo net, and being positioned against said torso of said individual;
two elongated body-height side cushions attached to said cargo net in a spaced-apart parallel relationship with said elongated body-height central cushion, one on each side of said elongated body-height central cushion and being respectively positioned along said cargo net against one of said shoulders and back of said individual;
handhold areas on said cargo net bordering said elongated body-height central cushion, and retaining said hands of said individual.

13. The nonviolent shield as claimed in claim 12, further comprising a plurality of handles attached to edges of said cargo net.

14. The nonviolent shield as claimed in claim 12, wherein said elongated body-height central cushion has a movable portion on an upper end thereof, for selectively moving said movable portion away from a plane of said elongated body-height central cushion and away from a head portion of said individual;

15. The nonviolent shield as claimed in claim 14, wherein said elongated body-height central cushion further comprises a hinge and said movable portion is attached to said central cushion along said hinge.

16. The nonviolent shield as claimed in claim 12, wherein said elongated body-height central cushion is about 12 inch wide and each of said handhold areas has a width of about 16 inches.

17. The nonviolent shield as claimed in claim 13, wherein each of said handles are attached to said cargo net by a Cow Hitch™ knot.

18. A method for restraining hands, torso, shoulders and back of an agitated standing individual comprising the steps of:
providing a flexible shield comprising an elongated body-height central cushion and two elongated body-height side cushions attached to a cargo net in a spaced-apart relationship;
mounting said elongated body-height central cushion along a median of said cargo net;
mounting said elongated body-height side cushions along said cargo net in such a way as to provide handholds areas on said cargo net on each side of said elongated body-height central cushion;
confronting said individual with said elongated body-height cushions positioned side-by-side in close proximity of each other and;
moving said flexible shield toward said individual while separating said elongated body-height cushions sideways from each other and exposing said handhold areas;
moving said handhold areas against said hands of said individual, while moving said elongated body-height central cushion against said torso of said individual;
wrapping said cargo net around said individual;
moving said elongated body-height side cushions against said shoulders and back of said individual, and
holding said elongated body-height cushions against said individual.

19. The method as claimed in claim 18, further comprising tying said cargo net behind said back of said individual.

20. The method as claimed in claim 18, further including the step of firmly holding a lower portion of said elongated body-height central cushion against said torso of said individual while lightly holding an upper portion of said elongated body-height central cushion against a head of said individual.
